(19) United States
(12) Patent Application Publication
(10) Pub. No.: US 2026/0056179 A1
(43) Pub. Date: Feb. 26, 2026

(54) METHOD IMPLEMENTED BY ARTIFICIAL INTELLIGENCE FOR PREDICTING A PERCENTAGE OF OIL

(71) Applicant: UNIVERSIDAD SIMÓN BOLÍVAR, Barranquilla, Atlántico (CO)

(72) Inventors: Reynaldo VILLARREAL GONZÁLEZ, Barranquilla, Atlántico (CO); Nataly GALÁN FREYLE, Barranquilla, Atlántico (CO); Leonardo PACHECO LONDOÑO, Barranquilla, Atlántico (CO); Samuel HERNÁNDEZ RIVERA, Mayaguez, PR (US); Juan PESTANA NOBLES, Barranquilla, Atlántico (CO)

(21) Appl. No.: 19/104,064

(22) PCT Filed: Aug. 16, 2023
(86) PCT No.: PCT/CO2023/000012

§ 371 Date: Feb. 14, 2025

(30) Foreign Application Priority Data
Aug. 17, 2022 (CO) NC2022/0011609

(51) Int. Cl. G01N 33/24; G01N 21/3563; G06F 18/15; G06F 18/2135; G06N 3/063; G06N 3/084; G06N 20/10

(52) U.S. Cl. CPC G01N 33/24; G01N 21/3563; G06F 18/15; G06F 18/2135; G06N 3/063; G06N 3/084; G06N 20/10

(57) ABSTRACT

The proposed solution addresses the identified problem by providing a method that involves the use of artificial intelligence (AI) in combination with a portable laser spectroscopy device, such as a quantum cascade laser, allowing in situ analysis.

### STATE OF THE ART

In the state of the art, there is the patent U.S. Pat. No. 4,528,508 ‘NUCLEAR MAGNETIC RESONANCE METHOD AND APPARATUS FOR REMOTE DETECTION AND VOLUMETRIC MEASUREMENT OF OIL RESERVES,’ which describes a new method and apparatus that allows for remote detection and in situ volumetric measurement of liquid oil reserves. A small fraction of the nuclear magnetic moments of the protons in a liquid oil reserve align with the Earth's magnetic field.

### FIELD OF THE INVENTION

The present invention belongs to the field of physical chemistry, particularly related to spectroscopy measurements for detecting specific spectra in matrices.

Unlike the solution found, the proposed invention does not require excavation, and detection is conducted in the field. Additionally, it does not require continuous scanning of the terrain; a single spectrometry measurement is sufficient to determine the presence of oil.

Another patent found is US2012271609 ‘Computing Methods and Systems for Hydrocarbon Exploration,’ which discloses computing methods and systems for hydrocarbon exploration. In one embodiment, an integrated petroleum system model is generated for an area of interest, based on a geological contour, a set of satellite remote sensing data, and a set of airborne remote sensing data.

The method used is entirely different from the one intended to be protected, as it employs LiDAR sensors for geological profiling and combines them with digital models of other data types to predict the presence of oil. This process requires extensive and complex preprocessing to prepare an enhanced geological map, and prediction is delayed as it depends on data analysis and processing within the method.

In this sense, the provided invention does not require any preprocessing or preparation, as the artificial intelligence used is pre-trained with multiple parameters before the field detection moment.

### TECHNICAL ADVANTAGES

The proposed solution offers several technical advantages, including the ability to perform field analysis immediately, without excavation, without requiring multiple scans or detection processes, and without preprocessing the analyzed soil sample. Additionally, the obtained data is processed by an artificial intelligence that instantly predicts the presence of oil in the matrix.

### BRIEF DESCRIPTION OF THE INVENTION

The proposed solution addresses the identified problem by providing a method that involves the use of artificial intelligence (AI) in combination with a portable laser spectroscopy device, such as a quantum cascade laser, allowing in situ analysis.

### BRIEF DESCRIPTION OF THE FIGURES

FIG. 1. Shows a flowchart of the process.

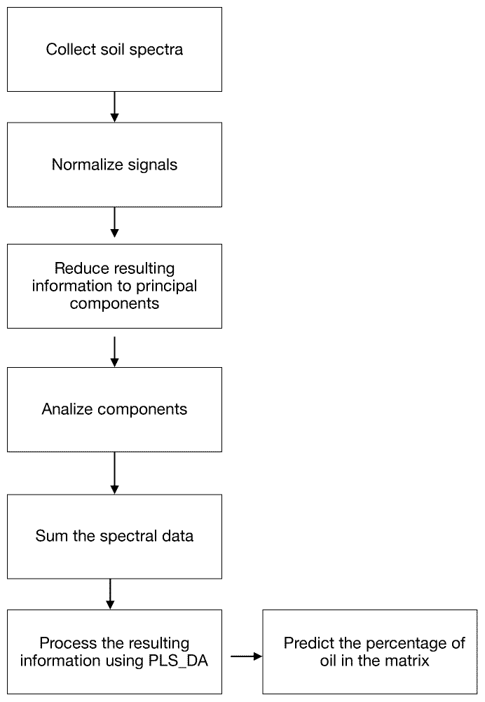

### DETAILED DESCRIPTION OF THE INVENTION

Current methods used to detect oil residues, particularly in soils, include spectroscopy and chromatography techniques that do not allow real-time measurement of residues and require prolonged detection times. These variables are critical when attempting to reverse soil contamination by oil residues or to identify oil presence in the field.

To address this, the provided solution comprises a method that combines high-power laser spectroscopy with data processing through artificial intelligence. This allows field data collection—the spectra—to be immediately analyzed by artificial intelligence hosted on a cloud server.

The artificial intelligence receives spectrometry data as input and outputs an indication of whether oil residues are present. The intelligence is pre-trained using a machine learning model that combines principal component analysis with a multivariable system to establish a multiple-variable system model with reduced-dimension data.

Spectroscopy data is fed into the system, which identifies patterns and determines the presence of petroleum contaminants in various soils.

The process includes collecting spectra from the soil to be analyzed. Spectroscopy must be performed at a point without vegetation and at an approximate distance of 15 cm. In a preferred embodiment of the invention, a quantum cascade laser (QCL) is used, although any high-power portable infrared spectroscopy device may be utilized.

The infrared spectrum of the soil is acquired over 5 seconds, and the data containing the spectroscopic signals is normalized through a vector normalization preprocessing step, which eliminates signal variance due to distance and light dispersion.

Preprocessed data undergoes multiple analysis phases using a multi-ensemble artificial intelligence model, which combines two machine learning models with a multilayer neural network.

To achieve this, the dimensionality of the normalized information is reduced to four components using principal component analysis. These components are analyzed using a support vector machine (SVM) model to determine soil type.

Subsequently, the information obtained from the SVM, along with the spectrum data, is processed using a machine learning model based on partial least squares discriminant analysis (PLS-DA). This enables the system to predict whether petroleum contamination is present.

If contamination is detected, a third semi-quantitative model is activated, predicting the oil percentage in predetermined concentration ranges present in the analyzed matrix. This model is part of a multilayer neural network comprising an input node, a hidden layer, and an output layer. The input node receives the processed information from the SVM and PLS-DA. The hidden layer processes the information using a compilation function and an Adam stochastic gradient descent optimization algorithm, while the output node reflects the predicted percentage of oil present in the analyzed matrix.

The entire system is installed on a cloud server that connects to a portable device receiving data from the spectroscopy apparatus. This enables real-time, in situ detection of contaminants.

What is claimed is:

1. A method for detecting oil, comprising the following phases:
Collecting soil spectra, where the collection can be performed using any high-power portable infrared spectroscopy device.
Normalizing the signals through a vector normalization preprocessing step.
Reducing the resulting information to four components through principal component analysis.
Analyzing the components using artificial intelligence, where the artificial intelligence employs a support vector machine learning model.
Adding the spectral data to the information obtained from the support vector machine learning model.
Processing the resulting information using a machine learning model based on partial least squares discriminant analysis.
Predicting the percentage of oil present in the analyzed matrix through a multilayer neural network.

2. The method of claim 1, wherein the multilayer neural network comprises an input node, a hidden layer, and an output node.

3. The method of claim 1, wherein the input node receives the information processed by the machine learning model based on partial least squares discriminant analysis.

4. The method of claim 1, wherein the hidden layer processes the information through a compilation function and an Adam stochastic gradient descent optimization algorithm.

5. The method of claim 1, wherein the output node reflects the prediction of the percentage of oil present in the analyzed matrix.
